(19) United States
(12) Patent Application Publication
(10) Pub. No.: US 2021/0277265 A1
(43) Pub. Date: Sep. 9, 2021

(54) CROSS-LINKABLE CELLULOSE AS 3D PRINTING MATERIAL

(71) Applicant: Stora Enso OYJ, Helsinki (FI)

(72) Inventors: Gisela Cunha, Nacka (SE); Heidi Saxell, Stockholm (SE); Robin Grankvist, Stockholm (SE)

(73) Assignee: Stora Enso OYJ, Helsinki (FI)

(21) Appl. No.: 17/251,360

(22) PCT Filed: Jun. 11, 2019
(86) PCT No.: PCT/IB2019/054843

§ 371 Date: Dec. 11, 2020

(30) Foreign Application Priority Data
Jun. 13, 2018 (SE) 1850728-5

(51) Int. Cl. C09D 11/14; C09D 11/106; C12N 5/00; C08B 15/00; B33Y 10/00; B33Y 70/00; B33Y 80/00

(52) U.S. Cl. CPC C09D 11/14; C09D 11/106; C12N 5/0068; B33Y 80/00; B33Y 10/00; B33Y 70/00; C08B 15/005

(57) ABSTRACT

A method for 3D printing is provided, using crosslinkable microfibrillated cellulose (MFC). The 3D printed structure is treated to provide crosslinking of the MFC.

The use of crosslinkable cellulose as a 3D printing material is provided.

### BACKGROUND

Microfibrillated cellulose (MFC) comprises partly or totally fibrillated cellulose or lignocellulose fibers. The liberated fibrils have a diameter less than 100 nm, whereas the actual fibril diameter or particle size distribution and/or aspect ratio (length/width) depends on the source and the manufacturing methods. The smallest fibril is called elementary fibril and has a diameter of approximately 2-4 nm (see e.g. Chinga-Carrasco, G., Nanoscale research letters 2011, 6:417), while it is common that the aggregated form of the elementary fibrils, also defined as microfibril, is the main product that is obtained when making MFC e.g. by using an extended refining process or pressure-drop disintegration process (see Fengel, D., Tappi J., March 1970, Vol 53, No. 3.). Depending on the source and the manufacturing process, the length of the fibrils can vary from around 1 to more than 10 micrometers. A coarse MFC grade might contain a substantial fraction of fibrillated fibers, i.e. protruding fibrils from the tracheid (cellulose fiber), with a certain amount of fibrils liberated from the tracheid (cellulose fiber).

There are different acronyms for MFC such as cellulose microfibrils, fibrillated cellulose, nanofibrillated cellulose, fibril aggregates, nanoscale cellulose fibrils, cellulose nanofibers, cellulose nanofibrils, cellulose microfibers, cellulose fibrils, microfibrillar cellulose, microfibril aggregrates and cellulose microfibril aggregates. MFC can also be characterized by various physical or physical-chemical properties such as large surface area or its ability to form a gel-like material at low solids (1-5 wt %) when dispersed in water.

MFC exhibits useful chemical and mechanical properties. Chemical surface modification of MFC has the potential to improve the properties of MFC itself, as well as products made from MFC, e.g. mechanical strength, water absorbance and—in certain circumstances—elasticity/flexibility.

Documents in this field include Lundahl et al. Ind. Eng. Chem. Res., 2017, 56 (1), pp 8-19, US 2016/214357, US 2004/038009, Markstedt et al. ACS Appl. Mater. Interfaces, 2017, 9 (46), pp 40878-40886 and Wang et al. Industrial Crops and Products Volume 109, 15 Dec. 2017, Pages 889-896.

Currently used materials for 3D printing are mainly thermoplastic polymers, resins, metals, ceramics and glass, which are predominantly non-degradable, derived from non-renewable resources, hydrophobic in nature and not necessarily biocompatible. Some exceptions exist, such as alginates and hydrophilic unmodified or chemically modified celluloses without crosslinking ability. To undergo crosslinking, these materials need external crosslinkers, such as cations or other reactive compounds that often are added in a multistep process. Consequently, utilizing such type of materials in 3D printing makes the process more complex.

There is therefore a need to provide alternative or improved materials and methods for 3D printing, as well as 3D printed structures comprising such materials. The 3D printed structures should have improved mechanical performance, in particular in terms of wet strength and—under certain conditions—flexibility.

### SUMMARY

It has surprisingly been found that is possible to use crosslinkable chemically modified cellulose such as phosphorylated cellulose or dialdehyde cellulose (DAC) as 3D printing material and after printing a two- or three dimensional structure, subject it to a post-treatment preferably heating, which triggers crosslinking, giving rise to 3D printed structures with significantly improved mechanical performance particular in terms of wet strength and under certain condition some elasticity.

A method for 3D printing is provided, comprising the steps of:
a. providing a composition comprising crosslinkable microfibrillated cellulose (MFC) wherein the crosslinkable MFC is phosphorylated microfibrillated cellulose (P-MFC) or dialdehyde microfibrillated cellulose (DA-MFC);
b. 3D printing said composition into a 3D structure;
c. treating said 3D structure to provide crosslinking of the MFC.

A 3D printed structure comprising crosslinked MFC is also provided. A 3D printer comprising a reservoir is also provided, wherein said reservoir contains a composition, preferably a suspension, comprising crosslinkable microfibrillated cellulose (MFC).

Further aspects of the invention are provided in the following text and in the dependent claims.

### DETAILED DISCLOSURE

In a first aspect, a method for 3D printing is provided, comprising the steps of:
a. providing a composition comprising crosslinkable microfibrillated cellulose (MFC) wherein the crosslinkable MFC is phosphorylated microfibrillated cellulose (P-MFC) or dialdehyde microfibrillated cellulose (DA-MFC);
b. 3D printing said composition into a 3D structure;
c. treating said 3D structure to provide crosslinking of the MFC.

In a first step of the method, therefore, a composition comprising crosslinkable MFC is provided. Microfibrillated cellulose (MFC) or so called cellulose microfibrils (CMF) shall in the context of the patent application mean a nano-scale cellulose particle fiber or fibril with at least one dimension less than 100 nm. MFC comprises partly or totally fibrillated cellulose or lignocellulose fibers. The cellulose fiber is preferably fibrillated to such an extent that the final specific surface area of the formed MFC is from about 1 to about 300 m²/g, such as from 1 to 200 m²/g or more preferably 50-200 m²/g when determined for a freeze-dried material with the BET method.

Various methods exist to make MFC, such as single or multiple pass refining, pre-hydrolysis followed by refining or high shear disintegration or liberation of fibrils. One or several pre-treatment steps are usually required in order to make MFC manufacturing both energy efficient and sustainable. The cellulose fibers of the pulp to be supplied may thus be pre-treated enzymatically or chemically, for example to reduce the quantity of hemicellulose or lignin. The cellulose fibers may be chemically modified before fibrillation, wherein the cellulose molecules contain functional groups other (or more) than found in the original cellulose. Such groups include, among others, carboxymethyl, aldehyde and/or carboxyl groups (cellulose obtained by N-oxyl mediated oxidation, for example “TEMPO”), or quaternary ammonium (cationic cellulose). After being modified or oxidized in one of the above-described methods, it is easier to disintegrate the fibers into MFC or NFC.

The nanofibrillar cellulose may contain some hemicelluloses; the amount is dependent on the plant source. Mechanical disintegration of the pre-treated fibers, e.g. hydrolysed, pre-swelled, or oxidized cellulose raw material is carried out with suitable equipment such as a refiner, grinder, homogenizer, colloider, friction grinder, ultrasound sonicator, single- or twin-screw extruder, fluidizer such as microfluidizer, macrofluidizer or fluidizer-type homogenizer. Depending on the MFC manufacturing method, the product might also contain fines, or nanocrystalline cellulose or e.g. other chemicals present in wood fibers or in papermaking process. The product might also contain various amounts of micron size fiber particles that have not been efficiently fibrillated.

MFC can be produced from wood cellulose fibers, both from hardwood or softwood fibers. It can also be made from microbial sources, agricultural fibers such as wheat straw pulp, bamboo, bagasse, or other non-wood fiber sources. It is preferably made from pulp including pulp from virgin fiber, e.g. mechanical, chemical and/or thermomechanical pulps. It can also be made from broke or recycled paper.

The above described definition of MFC includes, but is not limited to, the proposed TAPPI standard W13021 on cellulose nano or microfibril (CMF) defining a cellulose nanofiber material containing multiple elementary fibrils with both crystalline and amorphous regions, having a high aspect ratio with width of 5-30 nm and aspect ratio usually greater than 50.

A chemically-modified MFC comprising crosslinkable groups is thereby a crosslinkable MFC. Crosslinkable MFC forms bonds between the MFC upon treatment. Particular crosslinkable MFCs may be phosphorylated microfibrillated cellulose (P-MFC) or dialdehyde microfibrillated cellulose (DA-MFC); preferably P-MFC.

Phosphorylated microfibrillated cellulose (P-MFC) is typically obtained by reacting cellulose pulp fibers with a phosphorylating agent such as phosphoric acid, and subsequently fibrillating the fibers to P-MFC. One particular method involves providing a suspension of cellulose pulp fibers in water, and phosphorylating the cellulose pulp fibers in said water suspension with a phosphorylating agent, followed by fibrillation with methods common in the art. Suitable phosphorylating agents include phosphoric acid, phosphorus pentaoxide, phosphorus oxychloride, diammonium hydrogen phosphate and sodium dihydrogen phosphate.

In the reaction to form P-MFC, alcohol functionalities (—OH) in the cellulose are converted to phosphate groups (—OPO₃²⁻). In this manner, crosslinkable functional groups (phosphate groups) are introduced to the pulp fibers or microfibrillated cellulose.

Dialdehyde microfibrillated cellulose (DA-MFC) is typically obtained by reacting cellulose with an oxidising agent such as sodium periodate. During the periodate oxidation, selective cleavage of the C2-C3 bond of the anhydroglucose (AGU) unit of cellulose takes place, with concurrent oxidation of the C2- and C3-OH moieties to aldehyde moieties. In this manner, crosslinkable functional groups (aldehyde groups) are introduced to the cellulose.

The composition comprising crosslinkable MFC may be in the form of a suspension, a paste or powder comprising crosslinkable MFC. For ease of production and handling, the composition is preferably a suspension, more preferably an aqueous suspension of crosslinkable MFC.

In the case that the composition consists of crosslinkable MFC, no other components are present in the composition. In one aspect, said composition comprises more than 25%, preferably more than 50%, such as e.g. more than 75% by weight crosslinkable MFC. In one preferred embodiment, the composition may additionally comprise unmodified (native) MFC. Alternatively or additionally, the composition may additionally comprise other chemically-modified microfibrillated cellulose, such as TEMPO-MFC (i.e. MFC oxidised with 2,2,6,6-tetramethylpiperidin-1-yl)oxidanyl). The composition may comprise additional components, such as synthetic polymers, e.g. polyvinyl alcohol (PVOH), and/or inorganic fillers. This allows adjustment of the properties of the 3D printed structure.

According to one preferred aspect, the crosslinkable MFC is the only component of the composition which can crosslink. In such cases, the composition does not comprise additional crosslinking agents.

In the second step of the method, the composition is 3D printed into a 3D structure. Commercially-available 3D printers are suitable for use in such method steps.

In the third step of the method, the 3D structure is treated to provide crosslinking of the MFC.

When the crosslinkable MFC is phosphorylated microfibrillated cellulose (P-MFC), the treatment in step c is heat treatment, suitably at a temperature of between 60 and 200° C., preferably between 70 and 120° C. Heat treatment may take place via any known method, including blowing heated air, or placing the 3D printed structure into a heated environment, such as an oven or a heated platen.

When the crosslinkable MFC is dialdehyde microfibrillated cellulose (DA-MFC), the treatment in step c is reducing the pH, suitably to pH 7 or below, such as to pH 6 or below, or pH 5 or below.

In a preferred aspect, the 3D structure is treated while still in the 3D printing apparatus. As an alternative, the 3D structure may be removed from the 3D printing apparatus before treatment. Suitably, treatment takes place for a time of between 10 and 180 minutes.

Optionally, the method may further comprise the step of drying said 3D structure, before the treatment step. Drying can take place by any conventional means, e.g. drying in ambient temperature and RH.

The general steps of the method (3D printing, followed by treatment) may be carried out without any intervening method steps. Alternatively, one or more intervening method steps may be carried out between the 3D printing step and the treatment step.

If hydrated 3D printed structure is required, a further step of hydrating said structure with water after the treatment step may be carried out.

The present technology provides a 3D printed structure comprising crosslinked MFC. The presence of crosslinks between MFC fibrils can be ascertained by spectroscopic methods, e.g. ³¹P NMR in the case of P-MFC.

The 3D structures can exhibit high absorbency, flexibility and, under certain circumstances, also someelasticity. These characteristics make the crosslinkable cellulose a suitable material for 3D printing of structures requiring strong, flexible and hydrophilic material that also is biodegradable, renewable and biocompatible. Such structures can be useful in application areas such as hygiene, biomedical and food, and can span as an example from novel food to surgical implants.

In one aspect, the 3D printed structure described herein, and as made by the method described herein, may function as a biodegradable, biocompatible scaffold for growth of biological cells.

The 3D printed structure above may therefore further comprise one or more biological cells. The use of a 3D printed structure, as a scaffold for growth of biological cells, is also provided. Through 3D printing techniques, and selection of suitable crosslinkable MFC compositions, various regions of a 3D printed structure could be tailored to be preferential for growth and/or attachment of particular biological cells (e.g. due to a particular charge or pH of a region of a 3D printed structure).

A 3D printer comprising a reservoir, is also provided, wherein said reservoir contains a composition (preferably a suspension) comprising crosslinkable microfibrillated cellulose (MFC) as defined herein.

All details of the method for 3D printing (described above) are also relevant for the 3D printer and the 3D printed structure provided herein.

Although the invention has been described with reference to a number of aspects and embodiments, these aspects and embodiments may be combined by the person skilled in the art, while remaining within the scope of the present invention.

### SCHEMATIC DESCRIPTION OF THE FIGURES

FIG. 1: Shows a 2D top view of the human nose model printed with the different samples.

FIG. 2: Shows a 3D side view of the human nose model printed with the different samples (the lines represent the xx, yy, zz axes).

### EXAMPLE

3D printing of P-MFC and re-swelling capacity and properties of the 3D printed materials.

### Samples:

Aqueous dispersions of:
Enzymatically pre-treated native MFC (N-MFC; ^{˜}4% solids content)
Phosphorylated MFC (P-MFC; degree of functionalization=0.86 mmol/g; ^{˜}2% solids content; food grade green colorant)
Commercial bioink Cellink Xplore (according to manufacturer contains cellulose nanocrystals, alginate and coloring agent; ^{˜}16% solids content)

### Method:

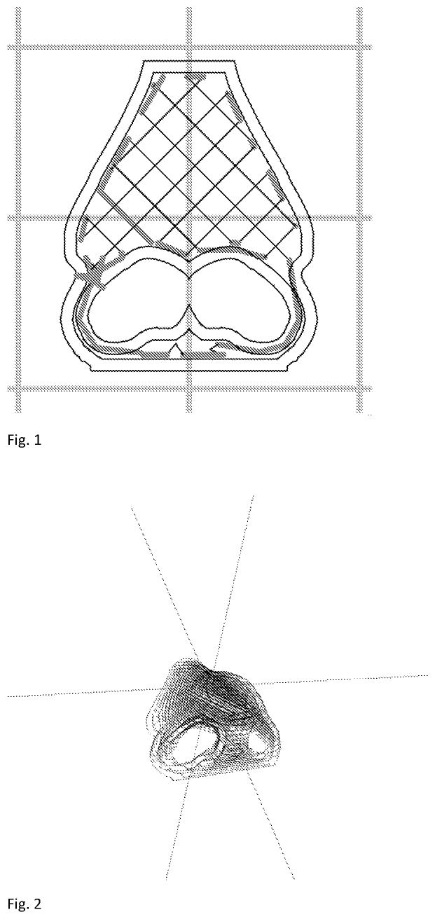

Human nose shapes (size: 15.05×19.23×8.50 mm; FIGS. 1 and 2) were 3D printed at RT with all the samples by using an Inkredible+ 3D bioprinter operating at a pressure in the range 50-70 kPa. Prior to printing, the samples were carefully loaded into 3 mL cartridges connected to conical nozzles (22G; made of polypropylene) to avoid air bubbles. The wet weight of the printed shapes was recorded after printing. They were then allowed to dry at the temperature and times listed in Table 1. In the case of the Cellink Xplore 3D printed shapes, a crosslinker solution consisting of 100 mM aqueous calcium chloride was added dropwise to some of the shapes immediately after printing. Three replicates were 3D printed and tested for each sample and drying conditions.

### Testing:

The samples were re-wetted after drying by soaking in deionized water for 20 minutes. The weight was recorded for the re-wet sample and the re-swelling capacity (or swelling recovery) was calculated as (w_{rw}/w_{iw})*100, where wᵣₑ stands for the weight of the re-wet sample and w_{iw} for the weight of the initial wet sample (prior to drying). Some properties of the 3D printed structures, namely compressibility, flexibility, elasticity and shape recovery, were then qualitatively and manually/visually assessed using a rating scale of 0-5, in which 0 means inexistent and 5 means very high. Compressibility was assessed by compressing the 3D printed shapes between the fingers; flexibility was assessed by manually bending the 3D printed shapes; elasticity was assessed by gently manually stretching the 3D printed shapes; shape recovery was visually assessed by comparing the shape of the re-wetted 3D printed shape after drying with the original wet shape.

### Results:

P-MFC dispersion, which comprised lower solids content than the benchmark materials (N-MFC and Cellink Xplore), proved to be a good bioink for 3D printing, and human nose 3D shapes were successfully printed. Crosslinked P-MFC-based 3D shapes (both dried at 70° C. and 105° C.) presented higher swelling recovery than the crosslinked Cellink Xplore bioink-based counterpart, as shown by the re-swelling capacity values. N-MFC-based 3D shapes presented the lowest re-swelling capacity, irrespectively of the drying conditions, likely due to an extensive degree of hornification upon drying, which is typical for unmodified MFC samples. Moreover, the re-wetted 3D shapes based on N-MFC didn't present any compressibility, flexibility or elasticity, and the shape recovery was extremely low. On the other hand, P-MFC-based 3D shapes were the ones demonstrating the highest compressibility, flexibility and elasticity, especially the one dried at 105° C. (highest crosslinking degree). Even though none of the 3D printed materials fully recovered the shape upon drying, the P-MFC-based 3D shape dried at 105° C. presented high shape recovery, similarly to the Cellink Xplore-based counterparts, indicating that the crosslinking of P-MFC without the addition of external crosslinkers is a viable route for the preparation of 3D printed materials with good performance.

TABLE 1
Drying conditions, re-swelling capacity and qualitative assessment of the
compressibility, flexibility, elasticity and shape recovery of the printed
3D shapes upon drying and subsequent re-swelling in water.

| | Drying | Drying | Re-swelling | Manual/Visual assessment (Rating scale: 0-5) | | | |
|---|---|---|---|---|---|---|---|
| | temperature | time | capacity | | | | Shape |
| Sample | (° C.) | (h) | (%) | Compressibility | Flexibility | Elasticity | recovery |
| P-MFC | RT | 48 | 36 ± 8 | 3 | 3 | 2 | 3 |
| | 70 | 2 | 55 ± 8 | 2 | 2 | 1 | 3 |
| | 105 | 0.83 | | | | | |
| | | (=50 | 44 ± 5 | 3 | 4 | 4 | 4 |
| | | min) | | | | | |
| N-MFC | RT | 48 | 14 ± 3 | 0 | 0 | 0 | 1 |
| | 70 | 2 | 15 ± 4 | 0 | 0 | 0 | 1 |
| Cellink | RT | 48 | 54 ± 4 | 0 | 1 | 0 | 4 |
| Xplore | | | | | | | |

1. A method for 3D printing, comprising the steps of:
a. providing a composition comprising crosslinkable microfibrillated cellulose (MFC), wherein the crosslinkable MFC is phosphorylated microfibrillated cellulose (P-MFC) or dialdehyde microfibrillated cellulose (DA-MFC);
b. 3D printing said composition into a 3D structure; and,
c. treating said 3D structure to provide crosslinking of the MFC.

2. The method according to claim 1, wherein the crosslinkable MFC is phosphorylated microfibrillated cellulose (P-MFC).

3. The method according to claim 1, wherein the composition comprising crosslinkable MFC is a suspension, a paste or powder comprising crosslinkable MFC.

4. The method according to claim 1, wherein said composition comprising crosslinkable MFC comprises more than 25%, by weight, crosslinkable MFC.

5. The method according to claim 1, wherein said composition comprising crosslinkable MFC further comprises at least one additional components.

6. The method according to claim 1, wherein the composition comprising crosslinkable MFC does not comprise additional crosslinking agents.

7. The method according to claim 1, wherein said crosslinkable MFC is phosphorylated microfibrillated cellulose (P-MFC), and wherein said treatment in step c is heat treatment at a temperature of between 60 and 200° C., preferably between 70 and 120° C.

8. The method according to claim 1, wherein said crosslinkable MFC is dialdehyde microfibrillated cellulose (DA-MFC), and wherein said treatment in step c is reducing a pH to a pH of 7 or below.

9. The method according to claim 1, wherein said treatment in step c takes place for a time of between 10 and 180 minutes.

10. The method according to claim 1, further comprising the step of drying said 3D structure, before the treatment in step c.

11. A 3D printed structure comprising crosslinked MFC.

12. The 3D printed structure according to claim 11, further comprising one or more biological cells.

13. The 3D printed structure according to claim 11, wherein the 3D printed structure comprises a scaffold for growth of biological cells and further comprises biological cells grown on the 3D printed structure.

14. The method according to claim 5, wherein the at least one additional components comprises a synthetic polymer, a polyvinyl alcohol (PVOH), or an inorganic filler.

15. The method according to claim 1, wherein the composition comprising crosslinkable MFC is an aqueous suspension.

16. The method according to claim 1 further comprising:
d. growing one or more biological cells on the 3D structure.
